(19) United States
(12) Patent Application Publication
(10) Pub. No.: US 2020/0388372 A1
(43) Pub. Date: Dec. 10, 2020

(54) METHOD AND APPARATUS FOR GENERATING DIETARY PREFERENCE BASED FOOD ITEM LISTINGS

(71) Applicant: Monginis Foods Pvt Ltd, Mumbai (IN)

(72) Inventor: Qusai Zoher KHORAKIWALA, Mumbai (IN)

(21) Appl. No.: 16/432,986

(22) Filed: Jun. 6, 2019

(51) Int. Cl. G16H 20/60; G06Q 30/06; G06Q 50/12

(52) U.S. Cl. CPC G16H 20/60; G06Q 99/00; G06Q 50/12; G06Q 30/0631

(57) ABSTRACT

The present invention provides a system, method and an apparatus for the generation of dietary preference based food item listings for users with dietary restrictions in a food ordering system. The system comprises of a set of databases i.e. user database, food item vendor database, a food items listing database and a dietary preferences database; a set of modules i.e. a dietary preference listings module, a food order request module, a calorific value preference module, a protein value preference module, a fat value preference module, a carbohydrate value preference module, an allergic restriction preference module and a dietary preference listings generator. With this invention, the dietary restricted user can view a subset of dietary preference based food item listings from multiple providers in a food ordering system.

### BACKGROUND OF THE INVENTION

The current online food ordering system environment includes members like ordering users, receiving users, food vendors, and hospitality service providers. Many online food ordering systems, and online food ordering mobile applications allow users to select food items from restaurant menus based on the location of the user and to get the ordered food delivered at the desired location. Recently, online food ordering system providers enable the live tracking of their food orders via a computer or mobile interface.

However, online food ordering systems, especially those offering online food ordering through mobile applications may not allow generation of food item listings from a variety of food providers and restaurants, based on the dietary preferences predefined by the vast majority of ordering users, partly due to the fact that the current online food ordering system and its computer-implemented method or computer implemented interface adaptations like food ordering mobile applications do not have the ability to allow ordering users to enlist dietary preferences particularly for food ordering platforms employing third parties such as, but not limited to stand-alone restaurants, caterers, food providers, street hawkers and franchised based food establishments.

Currently, there are systems and methods which provide a customized meal plan to the patients based on a chemotherapy treatment regimen. The customized meal plan is developed to improve health of the patient by alleviating the physical effects and loss in nutrition. The meal plan can be revised on the basis of further examination of the patients but is limited to only people advised/prescribed with this treatment regimen as disclosed in U.S. Pat. No. 6,083,006.

There are methods and systems available for calculating the daily calorie intake and to develop a food menu based on responses tendered by the participating user(s). The food menu prepared by employing such methods and systems provide an indication about the toxicity or deficiency levels of nutrients. In such case, the food menu prepared through a question-answer based evaluation and deals with immense time consumption as disclosed in US20150371553.

A user is restricted from ordering dietary-restriction limited food items from different food establishments based on predefined dietary preferences via dietary preference based food listings from multiple providers on a single food ordering platform or system wherein the dietary preferences include protein values, carbohydrate values, fat values and with allergic restrictions.

### SUMMARY OF THE INVENTION

The present invention specifically relates to generation of dietary preference based food item listings for users with dietary restrictions in a food ordering system.

Another aspect of the present invention is the generation of dietary preference based food item listings for users with dietary restrictions sorted by purchase price, location, delivery time or rating.

Yet another aspect of the present invention is generation of dietary preference based food item listings for users with dietary restrictions based on the calorific value, protein value, carbohydrate value, fat value and allergic restriction of the food item listing and associated predefined preference of the ordering user.

Other objects and advantages of the present invention will become apparent from the following description taken in connection with the accompanying drawings, wherein, by way of illustration and example, the aspects of the present invention are disclosed.

### BRIEF DESCRIPTION OF THE DRAWINGS

The disclosed embodiments have other advantages and features which will be more readily apparent from the detailed description, the appended claims, and the accompanying figures (or drawings). A brief introduction of the figures is below.

FIG. 1 is a functional block diagram of a food ordering system 100 according to one embodiment.

FIG. 2 is a functional block diagram of a dietary preference listings module 200 according to one embodiment.

FIG. 3 is a flow diagram of the method generation of dietary preference based food item listings for users with dietary restrictions in a food ordering system 100 according to one embodiment.

FIG. 4 illustrates modules of an example machine 400 able to read instructions from a machine-readable medium and execute them in a processor (or controller) according to one embodiment.

### DETAILED DESCRIPTION OF THE INVENTION

The following description describes various features and functions of the disclosed method and apparatus with reference to the accompanying figures. In the figures, similar symbols identify similar components, unless context dictates otherwise. The illustrative aspects described herein are not meant to be limiting.

These and other features and advantages of the present invention may be incorporated into certain embodiments of the invention and will become more fully apparent from the following description and claims or may be learned by the practice of the invention as set forth hereinafter. It may be readily understood that certain aspects of the disclosed system, method and apparatus can be arranged and combined in a wide variety of different configurations, all of which are contemplated herein.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a,” “an,” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

The Figures (FIGS.) and the following description relate to embodiments by way of illustration only. It should be noted that from the following discussion, alternative embodiments of the systems, methods, figures, diagrams and interfaces disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the systems, methods, figures, diagrams and interfaces illustrated herein may be employed without departing from the principles described herein. In the following description, for the purposes of explanation, numerous specific details are set forth in order to provide a thorough understanding of the various embodiments. It will be evident, however to one skilled in the art that the various embodiments may be practiced without these specific details.

### Configuration Overview

A system and method for generation of dietary preference based food item listings for users with dietary restrictions in a food ordering system is provided. The dietary preference listings module in the food ordering system may comprise a plurality of modules and generators including a food order request module, a calorific value preference module, a protein value preference module, a fat value preference module, a carbohydrate value preference module, an allergic restriction preference module and a dietary preference listings generator. The method for generation of dietary preference based food item listings for users with dietary restrictions in a food ordering system may comprise a plurality of steps receiving a dietary preference based food order request from users with dietary restrictions, determining the available food listings based on calorific value preference, protein value preference, fat value preference, carbohydrate value preference, or on allergic restriction preference and generating a subset of available dietary preference based food listings, and displaying the subset of available dietary preference based food listings to the users with dietary restrictions.

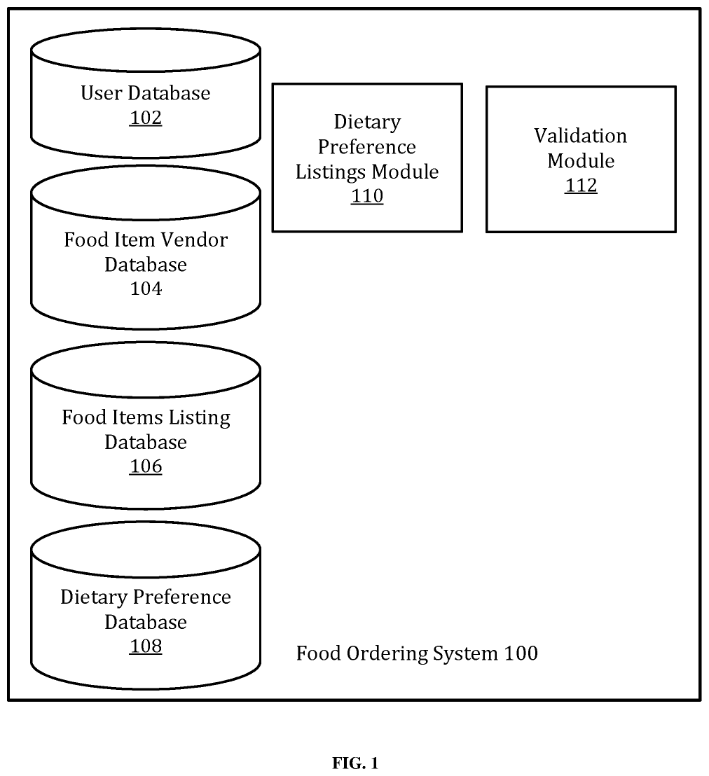

FIG. 1 is a functional block diagram of food ordering system according to an embodiment. A food ordering system 100 includes a plurality of databases and modules for generation of dietary preference based food item listings for users with dietary restrictions in a food ordering system 100. The dietary preference based food item listings are made available to users using the dietary preference listings module 200. Additional modules of the food ordering system 100 are described below.

A user database 102 may maintain information about each type of user based on the user's role in the system. The user information may be stored in a plurality of databases, each database associated with a user role, or the user roles may be stored in a single user database 102. For example, the additional user roles include users with dietary restrictions, food item vending users, order receiving users, and other types of users of the food ordering system 100.

The user database 102 is further configured for receiving, storing, updating and retrieving a plurality of data fields of each user, such as the user's name, address, and contact details. Depending on the user's role in the food ordering system 100, the user database 102 maintains additional information on the user. For example, for an exemplary ordering user, the user database 102 maintains ordering history in the food ordering system 100.

In one embodiment, a distinct food items vendor database 104 is configured for receiving, storing, updating and retrieving a plurality of data fields of each food vendors, comprising the name of the food establishment, location, special offers and discounts, delivery timings, preferences, food items with corresponding dietary values, and/or criteria if any, along with payment mode.

In one embodiment, a distinct food items listing database 106 is configured for receiving, storing, updating and retrieving a plurality of data fields of each food item listing, comprising the details of food items offered by each food establishment, specialty of the food item and availability of the food item, price, cuisine, and the associated dietary values of one serving of each such food item, the dietary values comprising the calorific value, the protein value, carbohydrate value, fat value, allergic restriction preference, glucose value, gluten value, type of diet compatibility (e.g. vegetarian diet) and other such dietary values in the corresponding unit of measurement (e.g. gms).

In one embodiment, a distinct dietary preference database 108 is configured for receiving, storing, updating and retrieving a plurality of data fields of each exemplary ordering user's predefined dietary preference, comprising the calorific value preference, protein value preference, carbohydrate value preference, fat value preference and allergic restriction preference with recommended daily, weekly or other time-bound values previously defined by each ordering user in the food ordering system 100.

A validation module 112 is configured for validating the user credentials of the exemplary users with dietary restrictions of the food ordering system 100.

A dietary preference listings module 200 is configured for generation of dietary preference based food item listings for a dietary restricted ordering user sorted by purchase price, location, delivery time or rating based on the dietary preference listings generation request received by an exemplary ordering user. In some embodiments, the generation of dietary preference based food item listings by the dietary preference listings module further comprises generation of dietary preference based food item listings based on the calorific value, protein value, carbohydrate value, fat value and allergic restriction of the food item listing and associated predefined preference of the ordering user.

In one embodiment, the user database 102, food item vendor database 104, food items listing database 106, dietary preference database 108 and other databases form a single consolidated database in the food ordering system 100.

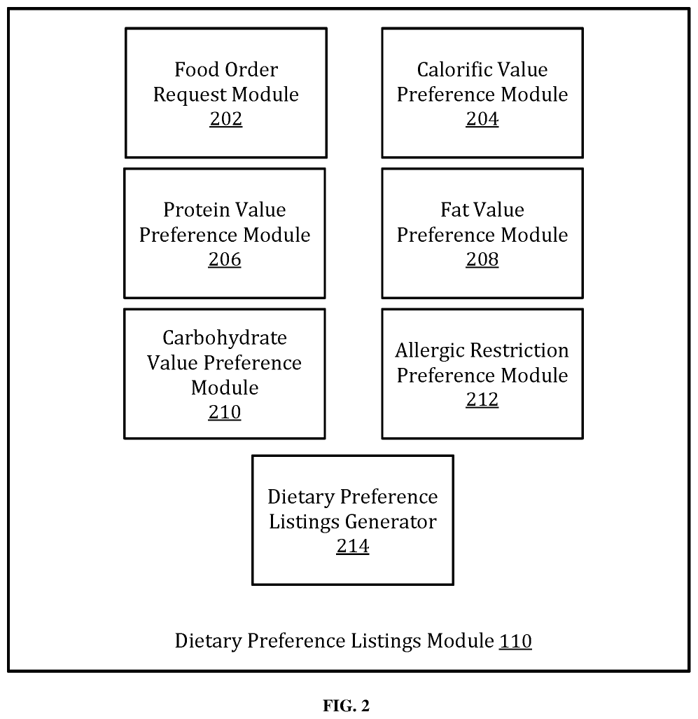

FIG. 2 is a block diagram of a dietary preference listings module 200 according to one embodiment. The dietary preference listings module 200 is configured for receiving, storing, retrieving and updating a plurality of dietary preference listings in the food ordering system 100. In some embodiments, the dietary preference listings may be accessed from a food items listing database 106.

The food order request module 202 is configured for receiving, storing, retrieving and updating a plurality of food order requests from exemplary food ordering users in the food ordering system 100.

The calorific value preference module 204 is configured for receiving, storing, retrieving and generating a plurality of calorific value preferences predefined by exemplary food ordering users in the food ordering system 100.

The protein value preference module 206 is configured for receiving, storing, retrieving and generating a plurality of protein value preferences predefined by exemplary food ordering users in the food ordering system 100.

The fat value preference module 208 is configured for receiving, storing, retrieving and generating a plurality of fat value preferences predefined by exemplary food ordering users in the food ordering system 100.

The carbohydrate value preference module 210 is configured for receiving, storing, retrieving and updating a plurality of carbohydrate value preferences predefined by exemplary food ordering users in the food ordering system 100.

The allergic restriction preference module 212 is configured for receiving, storing, retrieving and updating a plurality of allergic restriction preferences predefined by exemplary food ordering users in the food ordering system 100.

The dietary preference listings generator 214 is configured for receiving, storing, retrieving and updating a plurality of dietary preference based food item listings from a plurality of available food item listings based on the predefined dietary preferences of the exemplary user.

Although the dietary preferences listing module 200 is described as being composed of various modules, fewer or more modules (e.g. Trans Fat Value Preference Module, a Glucose Value Preference Module) may comprise the module with the present invention still falling within the scope of various embodiments.

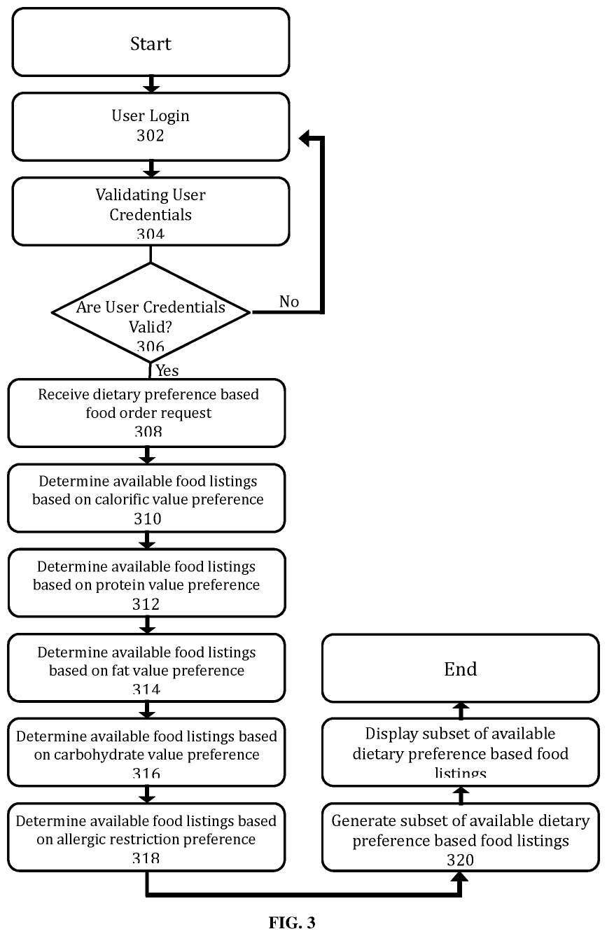

FIG. 3 is a flow diagram 300 of the method for generation of dietary preference based food item listings for users with dietary restrictions in a food ordering system 100 according to one embodiment. At step 302, an exemplary ordering user logs into the food ordering system 100 using an associated electronic user device. At step 304, the user credentials of the exemplary ordering user are validated. At step 306, provided the user credentials are valid, the exemplary user is allowed access to the food ordering system 100. If the user credentials are not valid, the exemplary user is denied access to the food ordering system 100.

At step 308, the dietary preference based food order request is received via the food order request module 202. At step 310, the calorific value preference is used to determine the availability of food item listings via the calorific value preference module 204. At step 312, the protein value preference is used to determine the availability of food item listings via the protein value preference module 206. At step 314, the fat value preference is used to determine the availability of food item listings via the fat value preference module 208. At step 316, the carbohydrate value preference is used to determine the availability of food item listings via the carbohydrate value preference module 210. At step 318, the allergic restriction preference is used to determine the availability of food item listings via the allergic restriction preference module 212.

At step 320, the subset of available dietary preference based food item listings is generated via the dietary preference listings generator 214, which is a subset of all the available food item listings. At step 322, the subset of available dietary preference based food item listings is displayed via the dietary preference listings module 200.

Although the food ordering system 100 is described as being composed of various components like databases and modules, the food ordering system 100 may comprise fewer or more databases, components, and other modules. For example, the food ordering system 100 may include a Trans Fat Value Preference Module, a Glucose Value Preference Module or a Gluten Value Preference Module with the present disclosure still falling within the scope of various embodiments. In some embodiments, an individual or group may play a plurality of user roles on the food ordering system 100, with the present disclosure still falling within the scope of various embodiments.

Yet another embodiment of the food ordering system 100 may comprise a web application, a mobile application, or an embedded module or subsystem of an online food ordering environment, a mobile food ordering environment, an electronic commerce system, an electronic payments system, a mobile operating system, a computer based operating system, or of a tablet based operating system, with the present disclosure still falling within the scope of various embodiments.

It is appreciated that, in some embodiments, various databases like 102, 104, 106, 108; modules 112, 200, 202, 204, 206, 208, 210, 212, 214 as well as the databases, engines, modules and components of the above embodiments may be stored in the form of machine readable instructions in the memory of the food ordering system 100 and executed by a processor 402 of the food ordering system 100 to perform one or more embodiments disclosed herein. Alternatively, the various databases like 102, 104, 106, 108, modules 112, 200, 202, 204, 206, 208, 210, 212, 214 as well as the databases, engines, modules and components of the above embodiments may be implemented in the food ordering system 100 in the form of an apparatus configured to perform one or more embodiments disclosed herein.

The online food ordering system environment includes a food ordering system 100 according to one embodiment. The food ordering system 100 operates in the online food ordering system environment and communicates with a plurality of exemplary user devices over an exemplary network. The exemplary user devices are operated by a plurality of kinds of users in the online food ordering system environment. The exemplary user devices may comprise any of a variety of computing devices, such as a desktop computer, a laptop, a mobile device, a tablet computer, a set-top box, a kiosk, interactive television, gaming console, and other computing platforms suitable for communicating with food ordering system 100. The food ordering system 100 provides a system for generation of dietary preference based food item listings for a dietary restricted ordering user. The food ordering system 100 enables the various users to communicate and transact with other users in an online food ordering system environment and provide services to other users. The exemplary network includes a wireless area network, a local area network, a General Packet Radio Service (GPRS) network, an Enhanced Data for Global Evolution (EDGE) network and the like. The exemplary user devices are connected to the food ordering system 100 via an exemplary network.

Although the online food ordering system environment is described as being composed of various, user devices (e.g., personal computer), a network (e.g., internet, intranet, world wide web), it would be appreciated by one skilled in the art that fewer or more kinds of users, user devices (e.g., a mobile phone device, a portable gaming console device, a tablet device, a learning console device, gaming console device or server device attached to a television or other screen) and networks (e.g., an intranet at hospitality service providers, restaurants, fashion products ordering platforms, FMCG, groceries and essential products ordering platforms and local travel booking platforms) may comprise the online food ordering system environment, with the present disclosure still falling within the scope of various embodiments.

### Computing Machine Architecture

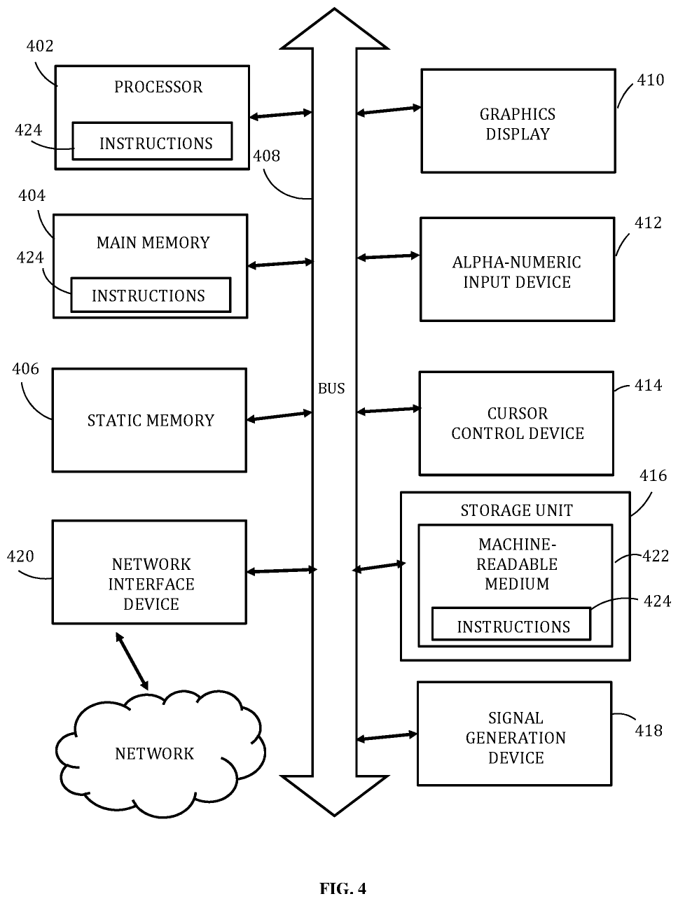

FIG. 4 is a block diagram illustrating modules of an example machine suitable for use as a food ordering system 100, in which any of the embodiments disclosed herein may be performed, according to one embodiment. This example machine is able to read instructions from a machine-readable medium and execute them in a processor 402 (or controller).

Specifically, FIG. 4 shows a diagrammatic representation of a machine in the example form of a computer system 400 within which instructions 424 (e.g., software) for causing the machine to perform any one or more of the methodologies discussed herein may be executed. In alternative embodiments, the machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The machine may be a server computer, a client computer, a personal computer (PC), a tablet PC, a set-top box (STB), a personal digital assistant (PDA), a cellular telephone, a smartphone, a web appliance, a network router, switch or bridge, or any machine capable of executing instructions 424 (sequential or otherwise) that specify actions to be taken by that machine. Further, while only a single machine is illustrated, the term “machine” shall also be taken to include any collection of machines that individually or jointly execute instructions 424 to perform any one or more of the methodologies discussed herein.

The example computer system 400 which carries out the computer-implemented method includes a processor 402 (e.g., a central processing unit (CPU), a graphics processing unit (GPU), a digital signal processor (DSP), one or more application specific integrated circuits (ASICs), one or more radio-frequency integrated circuits (RFICs), or any combination of these), a main memory 404, and a static memory 406, which are configured to communicate with each other via a bus 408. The computer system 400 may further include a graphics display unit 410 (e.g., a plasma display panel (PDP), a liquid crystal display (LCD), a projector, or a cathode ray tube (CRT)). The computer system 400 may also include alphanumeric input device 412 (e.g., a keyboard), a cursor control device 414 (e.g., a mouse, a trackball, a joystick, a motion sensor, or other pointing instrument), a storage unit 416, a signal generation device 418 (e.g., a speaker), and a network interface device 420, configured to communicate via the bus 408.

The storage unit 416 includes a machine readable medium 422 on which is stored instructions 424 (e.g., software) embodying any one or more of the methodologies or functions described herein. The instructions 424 (e.g., software) may also reside, completely or at least partially, within the main memory 404 or within the processor 402 (e.g., within a processor's cache memory) during execution thereof by the computer system 400, the main memory 404 and the processor 402 also constituting machine-readable media. The instructions 424 (e.g., software) may be transmitted or received over an exemplary network via the network interface device 420.

While machine readable medium 422 is shown in an example embodiment to be a single medium, the term “machine-readable medium” should be taken to include a single medium or multiple media (e.g., a centralized or distributed database, or associated caches and servers) able to store instructions (e.g., instructions 424). The term “machine-readable medium” shall also be taken to include any medium that is capable of storing instructions (e.g., instructions 424) for execution by the machine and that cause the machine to perform any one or more of the methodologies disclosed herein. The term “machine-readable medium” includes, but not be limited to, data repositories in the form of solid-state memories, optical media, and magnetic media.

The food ordering system 100 may be one or more servers in which one or more methods disclosed herein are performed. The processor 402 may be a microprocessor, a state machine, an application specific integrated circuit, a field programmable gate array, etc. (e.g., Intel® Pentium® processor). The main memory 404 may be a dynamic random access memory and/or a primary memory of the food ordering system 100. The static memory 406 may be a hard drive, a flash drive, and/or other memory information associated with the food ordering system 100.

The bus 408 may be an interconnection between various circuits and/or structures of the food ordering system 100. The video display 410 may provide graphical representation of information on the food ordering system 100. The alphanumeric input device 412 may be a keypad, keyboard and/or any other input device. The cursor control device 414 may be a pointing device such as a mouse.

The storage unit 416 may be a hard drive, a storage system, and/or other longer term storage subsystem. The signal generation device 418 may be a bios and/or a functional operating system of the food ordering system 100. The network interface device 420 may be a device that may perform interface functions such as code conversion, protocol conversion and/or buffering required for communication to and from a network. The machine readable medium 422 may provide instructions 424 on which any of the methods disclosed herein may be performed. The instructions 424 may provide source code and/or data code to the processor 402 to enable any one/or more operations disclosed herein. For example, the food ordering system 100 may be stored in the form of instructions 424 on a storage medium such as the main memory 404 and/or the machine readable medium 422 such as compact disk.

In one embodiment, a non-transitory computer-readable storage medium having tangibly embodied thereon a program of instructions executable by a processor 402 or a computing device (e.g., the food ordering system 100) causes the computing device to perform method steps illustrated in FIG. 3.

### Additional Configuration Considerations

Throughout this specification, plural instances may implement modules, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate modules in example configurations may be implemented as a combined structure or module. Similarly, structures and functionality presented as a single module may be implemented as separate modules. These and other variations, modifications, additions, and improvements fall within the scope of the subject matter herein.

Certain embodiments are described herein as including functionality implemented in computing logic or a number of components, modules, or mechanisms, for example, as illustrated in FIGS. 1, 2, and 3. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as Field Programmable Gate Array (FPGA) or an Application-Specific Integrated Circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors, e.g., processor 402, that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs)).

In another embodiment, the dietary preferences listing interface provided by the food ordering system 100 can be accessed over a local area network, intranet or virtual private network accessible to a limited plurality of user devices of hospitality service providers, restaurants, fashion products ordering platforms, FMCG, groceries and essential products ordering platforms and local travel booking platforms through a user device.

In another embodiment, dietary preferences listing interface provided by the food ordering system 100 can be accessed over a wide area network, General Packet Radio Service network, an Enhanced Data for Global Evolution network, a 3G telecommunications network, a 4G LTE telecommunications network or other telecommunications network through a user device.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Although the present embodiments have been described with reference to specific example embodiments, it will be evident that various modifications and changes may be made to these embodiments without departing from the broader spirit and scope of the various embodiments. For example, the various devices, modules, databases, etc. described herein may be enabled and operated using hardware circuitry (e.g., complementary metal-oxide-semiconductor (CMOS) based logic circuitry), firmware, software and/or any combination of hardware, firmware, and/or software (e.g., embodied in a machine readable medium).

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine modules that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

In addition, use of the “a” or “an” are employed to describe elements and modules of the embodiments herein. This is done merely for convenience and to give a general sense of the invention. This description should be read to include one or at least one and the singular also includes the plural unless it is obvious that it is meant otherwise.

According to the embodiments described in FIG. 1 through 3, various methods and electric structures may be embodied using transistors, logic gates, and electrical circuits (e.g., Application Specific Integrated Circuitry and/or in Digital Signal Processor circuitry). For example, the dietary preferences listing module and other modules of FIGS. 1 to 3 may be enabled using a dietary preferences listing circuit and other circuits using one or more of the technologies described herein. In addition, it will be appreciated that the various operations, processes, and methods disclosed herein may be embodied in a machine-readable medium and/or a machine accessible medium compatible with a data processing system (e.g., a server) and may be performed in any order. Accordingly, the specification and drawings are to be regarded in an illustrative rather than a restrictive sense.

Upon reading this disclosure, those of skill in the art will appreciate still additional alternative structural and functional designs for a system and a method for generation of dietary preference based food item listings for users with dietary restrictions in a food ordering system through the disclosed principles herein. Thus, while particular embodiments and applications have been illustrated and described, it is to be understood that the disclosed embodiments are not limited to the precise construction and modules disclosed herein. Various modifications, changes and variations, which will be apparent to those, skilled in the art, may be made in the arrangement, operation and details of the method and apparatus disclosed herein without departing from the spirit and scope defined in the appended claims.

1. A method for generating dietary preference based food item listings in a food ordering system, the system including one or more processors and a plurality of electronic user devices, the plurality of electronic user devices remotely linked over a computer network through a network interface device configured to perform functions enabling communication to and from the computer network via a mobile or browser-based web application, a computer desktop application, an electronic module or subsystem of an online food ordering environment, a mobile environment, an electronic commerce system, an electronic payments system, a mobile application or an Internet-based website, each of the plurality of user computing devices including an electronic user interface and an electronic display, the one or more processors configured with one or more computer-implemented modules or generators including a user preference module, a food item listing module, a validation module, a user database, a food item vendor database, a food items listing database, the method comprising the steps of:
receiving, via the food order request module, a dietary preference based food order request from an electronic device associated with at least one ordering user, the dietary preference based food order request comprising at least one location preference and at least one purchase price preference;
determining, via the calorific value preference module, at least one available food item listing from a plurality of food listings based on the predefined calorific value preference wherein the food item listing comprises a calorific value within the preferred range in the predefined calorific value preference of the user;
determining, via the protein value preference module, at least one available food item listing from a plurality of food listings based on the predefined protein macronutrient value preference wherein the food item listing comprises a protein macronutrient value within the preferred range in the predefined protein macronutrient value preference of the user;
determining, via the fat value preference module, at least one available food item listing from a plurality of food listings based on the predefined fat macronutrient value preference wherein the food item listing comprises a fat macronutrient value within the preferred range in the predefined fat macronutrient value preference of the user;
determining, via the carbohydrate value preference module, at least one available food item listing from a plurality of food listings based on the predefined carbohydrate macronutrient value preference wherein the food item listing comprises a carbohydrate macronutrient value within the preferred range in the predefined carbohydrate macronutrient value preference of the user;
determining, via the allergic restriction preference module, at least one available food item listing from a plurality of food listings based on the predefined allergic restriction preference wherein the food item listing fulfills the allergic restriction in the predefined allergic restriction preference of the user;
responding to the determination of available food listings, by
generating, via the dietary preference listings generator, a subset of dietary preference based food item listings from a plurality of available food item listings based on the dietary preference based food order request comprising the predefined dietary preferences of the ordering user;
displaying, via the dietary preference listings module, the subset of dietary preference based food item listings from a plurality of available food item listings based on the dietary preference based food order request comprising the predefined dietary preferences of the ordering user.

2. The method of claim 1, wherein the step of generating the subset of dietary preference based food item listings further comprises, displaying, via the dietary preference listings module, a subset of dietary preference based food item listings sorted by purchase price.

3. The method of claim 1, wherein the step of generating the subset of dietary preference based food item listings further comprises, displaying, via the dietary preference listings module, a subset of dietary preference based food item listings sorted by location.

4. The method of claim 1, wherein the step of generating the subset of dietary preference based food item listings further comprises, displaying, via the dietary preference listings module, a subset of dietary preference based food item listings sorted by delivery time.

5. The method of claim 1, wherein the step of generating the subset of dietary preference based food item listings further comprises, displaying, via the dietary preference listings module, a subset of dietary preference based food item listings sorted by rating.

6. A non-transitory computer readable storage medium having tangibly embodied thereon a program of instructions executable by a processor for generating dietary preference based food item listings in a food ordering system, the system including one or more processors and a plurality of electronic user devices, the plurality of electronic user devices being remotely linked over a computer network through a network interface device configured to perform functions enabling communication to and from the computer network via a mobile or browser-based web application, a computer desktop application, an electronic module or subsystem of an online food ordering environment, a mobile environment, an electronic commerce system, an electronic payments system, a mobile application or an Internet-based website, each of the plurality of user computing devices including an electronic user interface and an electronic display, the one or more processors configured with one or more computer-implemented modules or generators including a user preference module, a food item listing module, a validation module, a user database, a food item vendor database, a food items listing database, instructions when executed causing the processor to perform steps of:
receiving, via the food order request module, a dietary preference based food order request from an electronic device associated with at least one ordering user, the dietary preference based food order request comprising at least one location preference and at least one purchase price preference;
determining, via the calorific value preference module, at least one available food item listing from a plurality of food listings based on the predefined calorific value preference wherein the food item listing comprises a calorific value within the preferred range in the predefined calorific value preference of the user;
determining, via the protein value preference module, at least one available food item listing from a plurality of food listings based on the predefined protein macronutrient value preference wherein the food item listing comprises a protein macronutrient value within the preferred range in the predefined protein macronutrient value preference of the user;
determining, via the fat value preference module, at least one available food item listing from a plurality of food listings based on the predefined fat macronutrient value preference wherein the food item listing comprises a fat macronutrient value within the preferred range in the predefined fat macronutrient value preference of the user;
determining, via the carbohydrate value preference module, at least one available food item listing from a plurality of food listings based on the predefined carbohydrate macronutrient value preference wherein the food item listing comprises a carbohydrate macronutrient value within the preferred range in the predefined carbohydrate macronutrient value preference of the user;
determining, via the allergic restriction preference module, at least one available food item listing from a plurality of food listings based on the predefined allergic restriction preference wherein the food item listing fulfills the allergic restriction in the predefined allergic restriction preference of the user;
responding to the determination of available food listings,
generating, via the dietary preference listings generator, a subset of dietary preference based food item listings from a plurality of available food item listings based on the dietary preference based food order request comprising the predefined dietary preferences of the ordering user;
displaying, via the dietary preference listings module, the subset of dietary preference based food item listings from a plurality of available food item listings based on the dietary preference based food order request comprising the predefined dietary preferences of the ordering user.

7. The non-transitory computer readable storage medium of claim 6, wherein the step of generating the subset of dietary preference based food item listings executed by instructions further comprises, displaying, via the dietary preference listings module, a subset of dietary preference based food item listings sorted by purchase price.

8. The non-transitory computer readable storage medium of claim 6, wherein the step of generating the subset of dietary preference based food item listings executed by instructions further comprises, displaying, via the dietary preference listings module, a subset of dietary preference based food item listings sorted by location.

9. The non-transitory computer readable storage medium of claim 6, wherein the step of generating the subset of dietary preference based food item listings executed by instructions further comprises, displaying, via the dietary preference listings module, a subset of dietary preference based food item listings sorted by delivery time.

10. The non-transitory computer readable storage medium of claim 6, wherein the step of generating the subset of dietary preference based food item listings executed by instructions further comprises, displaying, via the dietary preference listings module, a subset of dietary preference based food item listings sorted by rating.

11. An apparatus comprising:
a network interface configured for generating dietary preference based food item listings in a food ordering system,
a processor coupled to the network interface;
a memory coupled to the processor, wherein the memory comprises instructions for execution on the processor to perform steps of:
receiving, via the food order request module, a dietary preference based food order request from an electronic device associated with at least one ordering user, the dietary preference based food order request comprising at least one location preference and at least one purchase price preference;
determining, via the calorific value preference module, at least one available food item listing from a plurality of food listings based on the predefined calorific value preference wherein the food item listing comprises a calorific value within the preferred range in the predefined calorific value preference of the user;
determining, via the protein value preference module, at least one available food item listing from a plurality of food listings based on the predefined protein macronutrient value preference wherein the food item listing comprises a protein macronutrient value within the preferred range in the predefined protein macronutrient value preference of the user;
determining, via the fat value preference module, at least one available food item listing from a plurality of food listings based on the predefined fat macronutrient value preference wherein the food item listing comprises a fat macronutrient value within the preferred range in the predefined fat macronutrient value preference of the user;
determining, via the carbohydrate value preference module, at least one available food item listing from a plurality of food listings based on the predefined carbohydrate macronutrient value preference wherein the food item listing comprises a carbohydrate macronutrient value within the preferred range in the predefined carbohydrate macronutrient value preference of the user;
determining, via the allergic restriction preference module, at least one available food item listing from a plurality of food listings based on the predefined allergic restriction preference wherein the food item listing fulfills the allergic restriction in the predefined allergic restriction preference of the user;
responding to the determination of available food listings,
generating, via the dietary preference listings generator, a subset of dietary preference based food item listings from a plurality of available food item listings based on the dietary preference based food order request comprising the predefined dietary preferences of the ordering user;
displaying, via the dietary preference listings module, the subset of dietary preference based food item listings from a plurality of available food item listings based on the dietary preference based food order request comprising the predefined dietary preferences of the ordering user.

12. The apparatus of claim 11, wherein the steps of generating the subset of dietary preference based food item listings executed by instructions further comprises displaying, via the dietary preference listings module, a subset of dietary preference based food item listings sorted by purchase price.

13. The apparatus of claim 11, wherein the steps of generating the subset of dietary preference based food item listings executed by instructions further comprises displaying, via the dietary preference listings module, a subset of dietary preference based food item listings sorted by location.

14. The apparatus of claim 11, wherein the steps of generating the subset of dietary preference based food item listings executed by instructions further comprises displaying, via the dietary preference listings module, a subset of dietary preference based food item listings sorted by delivery time.

15. The apparatus of claim 11, wherein the steps of generating the subset of dietary preference based food item listings executed by instructions further comprises displaying, via the dietary preference listings module, a subset of dietary preference based food item listings sorted by rating.
